(19) United States
(12) Patent Application Publication
(10) Pub. No.: US 2020/0375470 A1
(43) Pub. Date: Dec. 3, 2020

(54) GARMENT FOR MONITORING A USER AND METHOD FOR MAKING THE SAME

(71) Applicant: Siren Care, Inc., San Francisco, CA (US)

(72) Inventors: Jie FU, Shanghai (CN); Ran MA, San Francisco, CA (US); Henk Jan SCHOLTEN, The Hague (NL)

(21) Appl. No.: 16/997,835

(22) Filed: Aug. 19, 2020

Related U.S. Application Data
(63) Continuation of application No. PCT/US2019/018714, filed on Feb. 20, 2019.
(60) Provisional application No. 62/632,614, filed on Feb. 20, 2018.

(51) Int. Cl. A61B 5/01; A43B 3/00; A61B 5/00

(52) U.S. Cl. CPC A61B 5/01; A43B 3/0005; A43B 3/0031; A61B 5/0008; A61B 2562/16; A61B 5/447; A61B 2560/04; A61B 2562/0271; A61B 5/6807

(57) ABSTRACT

Systems for monitoring a user, and methods for manufacturing the same are described. Systems for monitoring a user may comprise a garment configured to be placed on a foot of a user comprising a conduit extending to a target sense location in a foot portion of the garment, one or more sensor assemblies each comprising a sensor arranged within the conduit, and a wireless communication module to communicate temperature data from a sensor to a computing device. Methods for manufacturing a system may comprise passing a sensor assembly into a conduit of a garment, the conduit extending to a foot portion of the garment, positioning the temperature sensor at a target sense location in the foot portion, coupling a distal portion of the sensor assembly to the garment to secure the sensor at the target sense location, and coupling a proximal portion of the sensor assembly to an electronics system.

### CROSS-REFERENCE TO RELATED APPLICATIONS

This application claims priority to U.S. Patent Application Ser. No. 62/632,614 filed on Feb. 20, 2018, which is hereby incorporated in its entirety by this reference.

### TECHNICAL FIELD

This invention relates generally to the field of foot care and more specifically to new and useful temperature-sensor garments and a method for making the same.

### BACKGROUND

Certain diseases may result in nerve damage, leading to decreased sensation in the extremities of a patient. For example, diabetes (an increasingly common medical condition in which the body has an impaired ability to produce or respond to the hormone insulin) damages blood vessels and nerves, particularly in the feet, and can lead to severe medical complications that are difficult to treat. For example, one complication of poorly controlled diabetes is foot ulcers, which may fail to heal because of poor blood circulation in diabetics and because treatment does not always successfully halt the spread of infection. Diabetic foot ulcers are painful and, when unresolved, can lead to lower limb amputations. As another example, Charcot foot, also known as Charcot arthropathy, is a debilitating complication of diabetes involving fractures and dislocations of bones and joints that occur with minimal or no known trauma. Diabetics may also suffer from diabetic neuropathy (numbness or less of feeling as a result of nerve damage), which results in decreased sensation in the feet.

Self-care is critical to detecting early signs of ulcers, Charcot foot, and other injuries and allowing timely treatment. However, decreased sensation in the feet due to diabetic neuropathy may impair the ability to self-detect these injuries. Further, visual inspection for detecting such conditions has limitations. For example, obese or visually impaired patients may not be able to see their feet easily. Even further, X-rays are unable to reliably show early stages of fractures. Accordingly, painful and dangerous foot conditions may be detected only when they have progressed to a more severe state, which increases the likelihood of extreme treatments such as amputation. Current methods of detecting early signs of ulcers, Charcot foot, and other injuries are limited to self-inspection and periodic check-ups by a physician. As described above, self-inspection may be severely impaired for diabetic patients, and doctor visits are time consuming, inconvenient, and may not be a reliable method of early detection.

Thus, there is a need for new and improved systems and methods for monitoring the feet health of patients.

### SUMMARY

Generally, a system for monitoring a user may include a garment configured to be placed on a foot of a user. The garment may include at least one conduit extending to a target sense location in a foot portion of the garment, at least one sensor assembly arranged at least partially in the conduit wherein the at least one sensor assembly comprises a temperature sensor located at the target sense location, and a wireless communication module configured to communicate temperature data from a temperature sensor to a computing device.

In some variations, the garment may be a sock, slipper, or the like, and include a leg portion, where a first portion of the conduit may be in the leg portion of the garment and a second portion of the conduit may be in the foot portion. The second portion of the conduit may, in some variations, include a plurality of channels diverging from the first portion of the conduit. Furthermore, the garment may include a plurality of sensor assemblies, at least some of which may be arranged at least partially in the conduit. In some of these variations, the plurality of channels may include a first channel housing a first sensor assembly and a second channel housing a second sensor assembly. In some variations, the first channel may direct temperature sensors of the first sensor assembly to a first target sense location, and the second channel may direct a temperature sensor of the second sensor assembly to a second target sense location.

The garment may include, in some variations, a first subset of channels extending along a left side of the foot portion, and a second subset of the channels extending along a right side of the foot portion. The garment may include a plurality of target sense locations arranged in a pattern on the sole region of the foot portion of the garment. In some variations, at least one target sense location may be an Ossa digit region of the garment, at least one target sense location may be between a phalange region and a metatarsal region of the garment, at least one target sense location may be between the metatarsal region and a tarsal region of the garment, and at least one target sense location may be in a heel region of the garment.

In some variations, the garment may include a compartment housing excess length of the at least one sensor assembly. The sensor assembly may include a proximal end, a distal end comprising a sensor, and at least one electrical lead extending between the proximal end and the sensor. In some variations, the sensor assembly may further include at least on axial fiber aligned with at least one electrical lead. Further, the sensor assembly may include at least one wrapping fiber surrounding the at least one electrical lead and the at least one axial fiber. In some variations, the proximal end of the sensor assembly may be coupled to an electronics system via the one or more electrical leads. In some variations, the sensor assembly may further include one or more engagement features which may be used to help position and/or anchor at least a portion (e.g., sensor) of the sensor assembly. For example, such an engagement feature may include a loop portion coupled to the distal end of the sensor assembly. The loop portion may be coupled to the garment proximate the target sense location. The loop portion may include an axial fiber formed into a loop that extends distal to the sensor. In some variations, the garment may further include a pocket. The pocket may contain an electronics system encased in a housing, wherein the electronics system includes at least one of a power source, a controller, and the wireless communication module.

Generally, a method of making a garment for monitoring a user may include passing at least one sensor assembly at least partially into a conduit of a garment, the conduit extending to a foot portion of the garment, wherein the sensor assembly includes a temperature sensor and/or other suitable sensor. The method may further include positioning the temperature sensor in the conduit at a target sense location in the foot portion of the garment. The method may further include coupling a distal portion of the at least one sensor assembly to the garment such that the temperature sensor is secured at the target sense location. The method may further include coupling a proximal portion of the at least one sensor assembly to an electronics system. In some variations, passing the at least one sensor assembly into the conduit may include passing the at least one sensor assembly through a first portion of the conduit in a leg portion of the garment, and into a second portion of the conduit in the foot portion of the garment. The conduit may include a plurality of channels diverging from the first portion of the conduit. In some variations, passing the at least one sensor assembly into the second portion of the conduit may include passing the at least one sensor assembly into one of the plurality of channels. In some variations, the garment may include a plurality of target sense locations arranged in a pattern on a sole region of the foot portion of the garment. The garment may, for example, include at least one target sense location in an Ossa digit region of the garment, at least one target sense location between a phalange region and a metatarsal region of the garment, at least one target sense location between the metatarsal region and a tarsal region of the garment, and at least one target sense location in a heel region of the garment. In some variations, the at least one sensor assembly may comprise an engagement feature such as a loop portion coupled to the distal end of the at least one sensor assembly, and coupling the distal portion of the at least one sensor assembly may include coupling the loop portion to the garment proximate the target sense location. The method may include substantially sealing the electronics system in a housing. The method may include placing the housing into a pocket of the garment. The method may further include shrinking the garment around the housing.

### BRIEF DESCRIPTION OF THE DRAWINGS

FIGS. 1A and 1B are schematic representations of the components of exemplary variations of a system for monitoring a user.

FIG. 2 depicts an exemplary variation of a garment for monitoring a user.

FIG. 3 depicts a cross-sectional view of the garment depicted in FIG. 2, taken along Plane A.

FIG. 4 depicts a cross-sectional view of the garment depicted in FIG. 2, taken along Plane B.

FIG. 5 depicts a cross-sectional view of the garment depicted in FIG. 2, taken along Plane C.

FIGS. 6A, 6B, and 6C depict detailed views of an exemplary variation of a sensor assembly.

FIG. 7 depicts a cross-sectional view of the garment depicted in FIG. 2, taken along Plane D.

FIGS. 8A and 8B depict a perspective bottom view and a bottom view, respectively, of a system for monitoring a user.

FIG. 9 is a schematic representation of an exemplary variation method of manufacturing a system for monitoring a user.

### DETAILED DESCRIPTION

Non-limiting examples of various aspects and variations of the invention are described and illustrated in the accompanying drawings.

Generally, various systems for monitoring a user and methods for making such systems are described. The system may be a garment (e.g., sock) worn on the foot of a user and configured to measure one or more physical characteristics of the user, such as skin temperatures at one or more locations on a foot of the user. In some variations, a user may wear two garments, including one garment on a left foot of the user, and another garment on a right foot of the user. Temperature measurements may be performed substantially continuously as the garments are worn.

For example, a diabetic user may suffer from diabetic neuropathy and consequently experience little or no feeling in the user's feet, which limits the user's ability to identify development of injuries such as sores, ulcers, infection, or poor blood circulation in the user's feet. If left untreated, such conditions may lead to greater medical complications, such as amputation of one or both feet. However, when a region of a foot becomes infected, the temperature of the affected region may generally rise as the body combats the infection. Accordingly, systems, when worn, can provide temperature data that may be analyzed to assess inflammation in diabetic feet and identify and/or detect development of diabetic foot complications. In some variations, a sampled temperature differential between corresponding locations on left and right feet of the user can be compared to a baseline temperature differential between the same corresponding locations. A threshold change between the baseline and sampled temperature differential (e.g., a change of about 4° F. or more) can indicate, for example, an early sign of diabetic foot ulcers. Exemplary methods for assessing foot inflammation based on foot temperature measurements from the system are described in U.S. Patent Publication No. 2017/0188841, which is hereby incorporated in its entirety by this reference.

Systems such as those described herein can provide substantially continuous temperature measurements at various locations of the user's feet, which enable a more effective prediction and/or detection of diabetic foot conditions compared to conventional methods of assessment. Conventional methods for monitoring diabetic foot conditions include using a handheld foot thermometer to measure temperature at selected locations of the foot. Such tools are designed to measure temperatures once a day or at long intervals. However, these tools fail to provide a comprehensive temporal and spatial understanding of temperature pattern data. In contrast, continuous monitoring allows the assessment of temperature over longer periods and with more temporal resolution, such that micropatterns (e.g., over the course of an hour or through the day) can be taken into consideration when assessing for development of foot conditions in the user. For example, a once-a-day measurement may capture a temporarily normal-looking temperature characteristic of a user's foot, but fail to capture subsequent signs of inflammation later in the day as the result of the day's activities. In contrast, continuous monitoring can facilitate analysis of temperature patterns (both spatial and temporal) specific to a user, for more accurate assessments. Furthermore, the systems herein may incorporate activity data (e.g., from an accelerometer, pedometer, etc.) such that analysis can take into consideration varying levels of user activity over time (and assess how activity affects temperature patterns). Accordingly, continuous monitoring has a greater potential to report consistent and clinically relevant temperature increases. Thus, systems which provide continuous temperature monitoring have the potential to further improve home care and early detection of diabetic foot conditions. Variations of such systems, and methods for making such systems, are described in further detail below.

### Systems for Monitoring a User

Systems for monitoring a user may generally comprise a garment, one or more sensor assemblies each comprising a sensor, and an electronics system coupled to the one or more sensor assemblies. In one exemplary variation, the garment may be configured to be placed on the foot of a user. The garment may comprise one or more conduits extending to a target sense location in a foot portion of the garment. The garment may also comprise one or more sensor assemblies, each comprising a temperature sensor located at a target sense location, arranged at least partially within the conduit. The garment may further comprise an electronics system configured to communication temperature data from the temperature sensors of the one or more sensor assemblies to a computer device.

In one exemplary variation, the garment may be configured to be worn on the foot of a user, and may comprise a leg portion and a foot portion comprising a sole. The garment may be configured to support an electronics system in the leg portion or other suitable portion of the garment, and sensors in one or more target sense locations across the sole of the foot portion. The leg portion of the garment may comprise a pocket configured to house at least a portion of the electronics system. The garment may further comprise a conduit, wherein one or more sensor assemblies may be situated within the conduit. A first portion of the conduit may be situated in the leg portion of the garment, and a second portion of the conduit may be in the foot portion of the garment. The second portion of the conduit may comprise one or more channels, which may extend from the first portion of the conduit to the one or more target sense locations in the sole of the foot portion. One or more sensor assemblies may extend from the first portion of the conduit and into the one or more channels. The channels may branch off from the first portion of the conduit, and each channel may direct the sensor of each sensor assembly to a target sense location on the sole of the foot portion. For example, a plurality of sensor assemblies may be arranged at least partially within the conduit, and the plurality of channels may comprise a first channel housing a first sensor assembly, and a second channel housing a second sensor assembly, wherein the first channel directs one sensor assembly to a first target sense location and the second channel directs one sensor assembly to a second target sense location.

In one exemplary variation, a sensor assembly may comprise a proximal end, a distal end comprising a sensor, and one or more electrical leads coupled to the sensor and extending between the proximal end and the sensor. When situated in the garment, the sensors of multiple sensor assemblies may be located at the target sense locations and form a pattern on the sole of the foot portion of the garment. This pattern may, for example, be optimized to target pressure points on the foot of the user. The sensors of the sensor assemblies may be temperature sensors, for example, but may additionally or alternatively include other sensors such as pressure sensors, etc. In one exemplary variation, the sensor may transmit temperature data from the user's foot to the electronics system. The electronics system may contain various suitable components such as a power source (e.g., battery), a processor, a controller, and/or a wireless communication module, at least some of which may be located on a printed circuit board (PCB). The electronics system may be encased in a housing.

Generally, the electronics system may power the one or more sensors, collect data from the one or more sensors, and offload the sensor data to an external computational device (e.g., for analysis). For example, the proximal end of each sensor assembly may be coupled to the electronics system via the electrical leads of the sensor assembly. The power source of the electronics system may supply power to the sensor. The controller may collect temperature data communicated by the sensor(s) at the distal end of the sensor assembly or assemblies. The wireless communication module may transmit the data collected by the controller to an external computational system. The PCB may support the controller, the power source, the wireless communication module, and/or any other components of the electronics system. The components of the electronics system may be encased in a housing. An adhesive or sealant may be applied to the housing. The sealant or adhesive may, for example, serve to waterproof the housing and/or assist in securing the connection of the sensor assemblies to the electronics system.

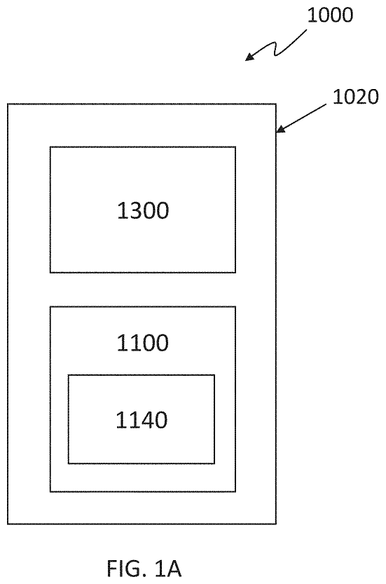

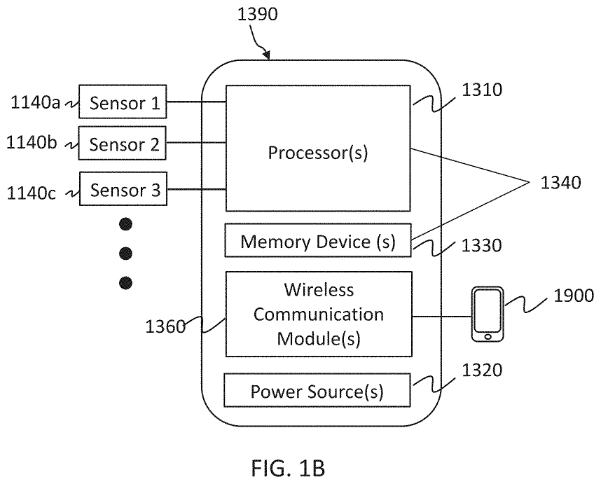

In general, systems for monitoring a user may comprise a garment configured to generate data, transmit the data from one location of the garment to a second location of the garment, and communicate the data to an external system for processing. FIG. 1A is a schematic representation of a portion of a system for monitoring a user. The system 1000 for monitoring a user may comprise at least one garment 1020, an electronics system 1300, and one or more sensor assemblies 1100, each sensor assembly comprising a sensor 1140. The sensor 1140 may be located at an end (e.g., distal end) of the sensor assembly 1100. The sensor 1140 of a sensor assembly 1100 may be a temperature sensor and may generate temperature data transmitted to the electronics system 1300 via the sensor assembly 1100. FIG. 1B is a schematic representation of sensors and other electronic components of a system for monitoring a user. In general, in some variations an electronics system may comprise at least one power source 1320, at least one processor 1310 and at least one memory device 1330 comprising at least one controller 1340 of the system, at least one wireless communication module 1360, and one or more sensors (1140a, 1140b, 1140c, etc.). Sensors 1140a, 1140b, and 1140c (and the like) may be coupled to the processor 1310. The controller may receive data from the sensors via the processor(s) 1310, and may locally store the data via the memory device 133. The power source 1320 may supply power to the sensors. The controller may offload the data to the wireless communication module, which may be configured to transmit data to an external computational system.

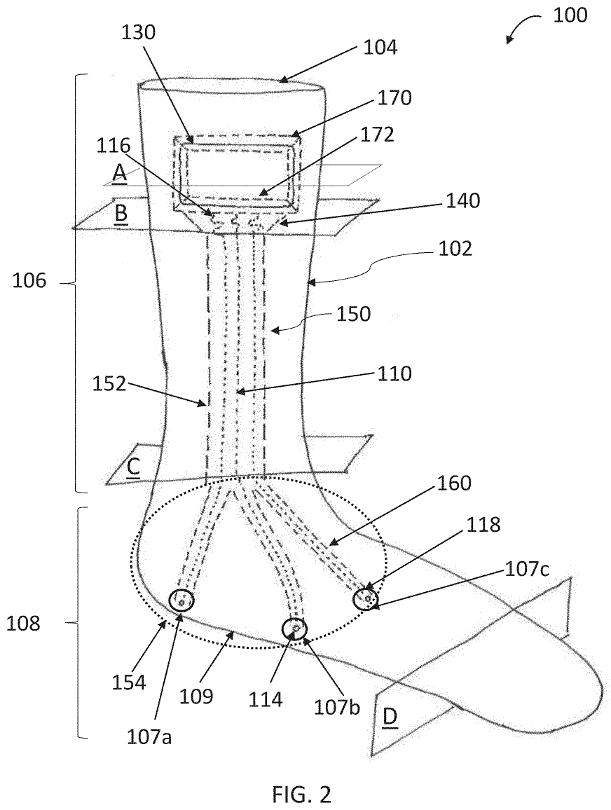

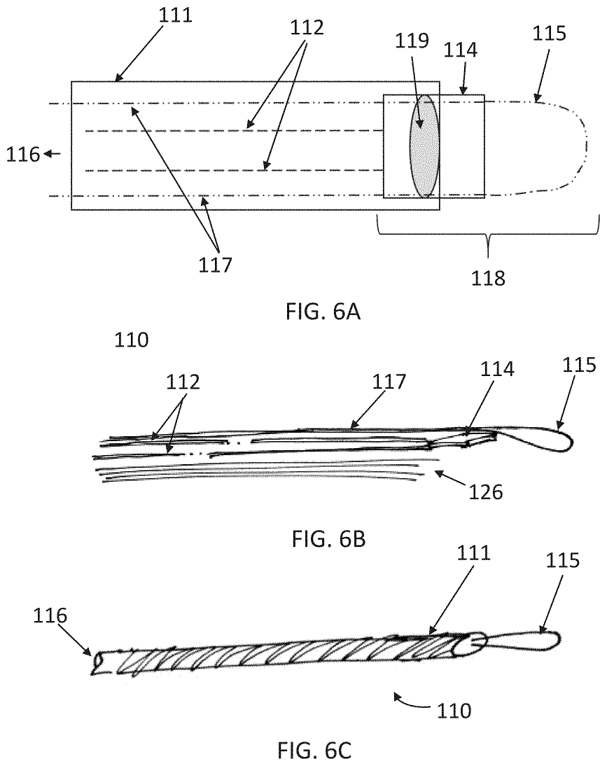

In some variations as shown in FIG. 2, a system 100 for monitoring a user may comprise at least one garment 102 configured to be worn on the foot of a user. The garment 102 may be configured to support components used to collect and interpret biometric data such as temperature data from target sense locations on the foot of a user. This data may be used to assess, for example, whether the user has a foot condition that requires attention. The garment may comprise one or more sensor assemblies 110 and an electronics system 130 coupled to the one or more sensor assemblies 110. The garment 102 may comprise various openings or channels to direct the sensor assemblies 110 from the electronics system 130 to target sense locations 107 in the garment where sensor measurements are desired to occur. In some variations, the garment 102 may comprise a mouth 104, a leg portion 106, and a foot portion 108 comprising a sole 109. The leg portion 106 of the garment 102 may comprise a pocket 170, a conduit 150, and a buffer region 140 as described in further detail below. The pocket 170 may be configured to house the electronics system 130. The sensor assemblies 110 may be connected to the electronics system 130 at a proximal end. The sensor assemblies 110 may extend from the pocket 170 into the conduit 150. The conduit 150 may partially house the sensor assemblies 110, and direct the sensor assemblies 110 to target sense locations 107 in the foot portion 108 of the garment 102. A first portion 152 of the conduit 150 may be contained within the leg portion 106 of the garment 102. A second portion 154 of the conduit 150 may be contained within the foot 108 portion of the garment 102. The second portion 154 of the conduit in the foot portion 108 may comprise one or more channels 160 directed to target sense locations 107 in the foot portion 108. The channels 160 may branch from the first portion 152 of the conduit 150 in the leg portion 106 to the target sense locations 107 in the foot portion 108. The sensor assemblies 110 may be directed from the pocket 170, through the first portion of the conduit 152, into the channels 160 of the second portion of the conduit 154, and terminate at the target sense locations 107. Each sensor assembly 110 may comprise a proximal end 116, a distal end 118 comprising a sensor 114, and electrical leads 112 extending from the proximal end 116 to the sensor 114. As shown in FIG. 2, each sensor assembly 110 may be directed by one channel 160 to a target sense location 107. The sensor 114 of the each sensor assembly may be positioned at a target sense location 107 in the foot portion 108. The sensor 114 at the distal end 118 of the sensor assembly 110 may be secured to the garment 102 within the channel 150 to keep the sensor 114 in place. As shown in FIGS. 6A and B, when positioned at the target sense locations 107, the sensors 114 may form a pre-determined pattern on the sole 109 of the foot portion 108.

### Garment

Generally, in some variations, the garment may provide a platform or other structural support for other components of the system (e.g., sensors, electronics system, etc.) and/or help position sensors relative to desired measurement locations of the user when the garment is worn. The garment 102 of the system 100 may be shaped to accommodate a foot of a user. However, other variations of the garment may be shaped to accommodate a different part of a user, such as a hand, for example. In some variations, the garment includes a sock configured to be placed or worn on a foot of the user. However, the garment may alternatively be any suitable component to be positioned on the foot such as a shoe, a slipper, an insole, etc. The garment may be configured specifically for a left foot (e.g., include a toe box accommodating contours of a left foot), for a right foot (e.g., include a toe box accommodating contours of a right foot), or may be universally or suitable for both feet. The garment may include one or more labels that are sewn, woven, or otherwise incorporated into or coupled to the garment. Examples of labels include an indication of left or right foot compatibility, size (e.g., small, medium, large, or numeric size), or other identifying info.

The garment 102 may be made of any suitable material. The material may, for example permit the user to stretch, strain and/or repeatedly the garment, and may be durable enough to withstand everyday wear for a substantial person of time. The garment may also provide the benefit of being comfortable for the user, and insulate the part of the user on which it is worn. Further, the garment may comprise an antimicrobial material (e.g., silver threads) or material treated with a suitable antimicrobial agent. The garment may, for example, be made of wool, cotton, nylon, polyester, or any other natural, synthetic, and/or blended fibers. Further, the garment may be constructed by any suitable method, such as knitting and/or weaving the fibers together. The garment may be constructed to support the components of a garment to monitor a user. The garment may be constructed to protect the components of the garment, and enable the sock to be dressed onto a foot, worn, undressed from the foot, washed, and folded repeatedly over a period of time. For example, the garment may be constructed from durable material that can withstand repeated wear and washing, as well as strain from being pulled on and off of the foot of a user.

In some variations, the garment 102 may comprise one or more markers to indicate which foot the garment corresponds to. Any suitable marker may be used to designate the footing of the garment. A marker may comprise a patch, stitching, color dye, variations in fiber material, etc. In some variations, a marker may be a visual marker and include one or more features such as a particular pattern, a particular letter, a particular color or shape (e.g., arrows), and the like. In one variation for example, an “L” or an “R” may be apparent on the fabric of the garment 102. In another variation, a color-coded marker may be used to designate which foot the garment corresponds to. For example, a left sock may comprise a blue marker, and a right sock may comprise a red marker. Additionally or alternatively, a marker may be a tactile marker and include a textured (e.g., raised) surface. The marker may be located on any suitable location of the garment. For example the marker may be located on a front, back, medial, or lateral surface of a leg or foot portion of the garment. Because the sensors 114 may be arranged in a left foot- or right foot-specific pattern on the sole 109 of the foot portion 108, it may be important that the user wear the garment 102 on the correct designated foot. Specifically, the sensor pattern may correspond to a specific left foot- or right foot-biased pattern of target sense locations on the foot of the wearer. Therefore, the sensors will not match up to the target sense locations if the sock is worn on the incorrect foot.

### Pocket

A garment 102 of a system 110 for monitoring a user may comprise at least one structure such as a pocket to house and support one or more electrical components 130 of the system. The pocket 170 may house electronics components used to collect and transmit temperature data as described in further detail below, and may facilitate the connection of sensor assemblies 110 to these electronic components. For example, the pocket 170 of a garment configured to be worn on the foot of user may comprise a pocket 170 in the leg portion 106 of the garment, as depicted in FIG. 2. In the variation depicted in FIG. 2, the pocket 170 in the leg portion 106 is distal to the mouth 104 of the garment 102, and proximal to the conduit 150. A pocket 170 arranged near the mouth 104 of the garment may provide the benefit of remaining out of the way of a shoe, boot, slipper, or other outerwear worn over the garment 102. Avoiding interference with outwear may aid in the comfort of the user, and/or may further avoid interference with wireless signals transmitted by the device. However, in other variations, the pocket 170 may be located elsewhere on the garment 102. For example, the pocket 170 may be located in the foot portion 108 (e.g., ankle region) of the garment 102. In the variation depicted in FIG. 2, the pocket 170 is configured to accept and retain the electronics system 130 encased in a housing 139. In FIG. 2, the pocket 170 and the housing of the electronics system 130 are shown as generally rectangular in shape, and the shape of the pocket 170 corresponds the shape of the housing 139. However, the pocket 170 and the housing 139 may be of any suitable shape, such as circular, elliptical, square, or triangular, etc. Further, the pocket 170 and the electronics system housing 139 need not be the same shape. As depicted in FIG. 2, the pocket 170 may be located on a lateral side of the garment such that the pocket and electronics systems fall on the outside of a user's lower leg when the sock is worn. That is, when the garment is worn on a user's left foot, the pocket may be located on the left side of the leg. With the pocket arranged in this way, when the user is wearing a garment on each of a left foot and a right foot, the electronics systems 130 on the left and right garments will not face each other. This may reduce the opportunity for the electronics systems 130 to contact one another while the user is partaking in daily activities such as walking, running, crossing her legs, or any other activity. Therefore, a pocket 170 located on a lateral side of the garment 102 may reduce the opportunities for the electronics systems to cause discomfort to the user. However, the pocket may be situated in any suitable location of the garment. For example, the pocket may be on the front or back surfaces of the garment.

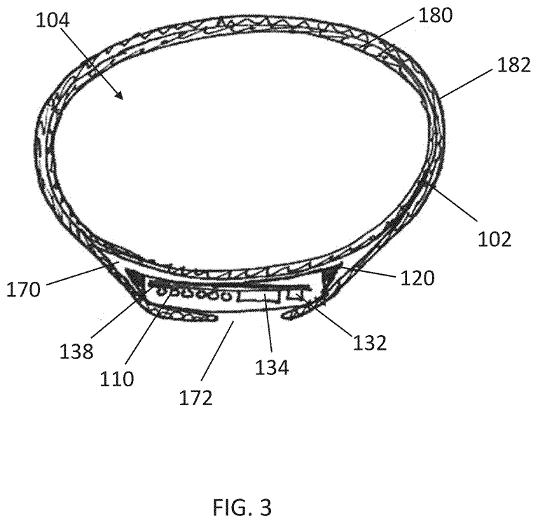

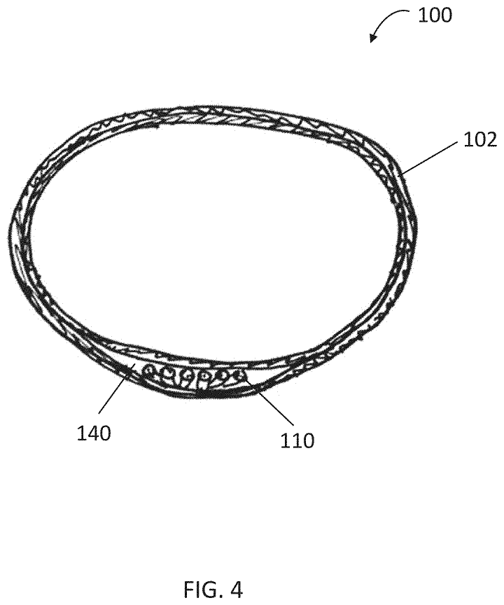

The pocket 170 may also comprise an opening configured to accept and/or retain the electronics system and housing. In some variations, the garment portion and the electronics system 130 may be manufactured separately, and the electronics system 130 may be situated in the pocket after the garment has been constructed. Thus, during manufacturing, the pocket 170 may provide the benefit of allowing the electronics system 130 to be installed in the garment 102 after the garment is manufactured. However, when the garment 102 is worn by a user, it may be beneficial for the pocket to be able to retain the electronics system and housing within the garment. In one variation of the system, an unwashed garment may comprise a pocket with an opening that the housing can enter through. The garment 102 may then be washed to shrink the opening 172 of the pocket 170 in order to retain the electronics system 130 within the pocket 170. As depicted in FIGS. 2 and 4, the opening 172 of the pocket may be configured to accept and/or retain the electronics system 130 and housing 139 within the garment. The opening may be shaped to accommodate the housing of the electronics system. The opening may be an opening in one layer of the garment 102 such that the components contained by the housed are situated between layers of the garment. The opening 172 depicted in the exemplary variation of FIGS. 2 and 3 is smaller than the housing of the electronics system 130. This may provide the benefit of retaining the housing and electronics system in the garment. The pocket 170 and opening 172 may be comprised of any suitable material. For example, the layers of the garment may be sewn together to define the pocket. Alternatively, the garment 170 may comprise two layers stitched together, and bridged with knitted fibers to form the pocket 170. In another variation, the pocket 170 may be constructed by sewing an additional layer of fabric to an internal or external surface of the garment 102. The pocket 170 may be comprised of any suitable material, and need not be comprised entirely of one material. For example, the pocket may be partially comprised of an elastic material to assist in retaining the electronics system 130. In some variations, the pocket 170 may comprise a mechanism to close the pocket and retain the electronics system 130 and housing 139, such as, for example, a zipper, an elastic rim, snaps, magnets, or any other suitable opening or closing mechanism. Furthermore, in some variations, the pocket may include flap or other suitable cover (or partial cover) that fully encloses the housing in the pocket. In some variations, the pocket 170 may not comprise an opening 172 for the electronics system 130. For example, the pocket may be sewn shut after the electronics system 130 is placed in the pocket 170. In another variation, the garment may not comprise a pocket (e.g., electronics system and/or housing may be attached directly to the garment using one or more fasteners). For example, in one variation, the housing may be attached to the garment using adhesive. In another variation, the housing may be sewn onto the garment. In another variation, the housing may be attached to the garment via magnets.

### Conduit

The garment may further comprise at least one conduit 150 that houses and directs one or more sensor assemblies 110 through the garment to target sense locations 107 in the garment. For example, at least some target sense locations may, in some variations, correspond to pressure points on the user's foot. Pressure points may be locations that tend to experience high pressure, such as when the user is ambulatory. For example, the conduit 150 may direct the sensor assemblies 110 from the pocket of the garment 102 to target sense locations 107 on the foot portion 108 of the garment. As depicted in the exemplary variation in FIG. 2, the conduit may traverse both the leg portion 106 and foot 108 portion of the garment 102. For example, a first portion 152 of the conduit 150 may be situated in the leg portion 106 of the garment 102, and a second portion 154 of the conduit 150 may be situated in the foot portion 106 of the garment 102. The first portion 152 of the conduit 154 may direct one or more sensor assemblies 110 along the leg portion 106 of the garment. For example, multiple sensor assemblies may be bundled and directed collectively within the first portion 152 of the conduit. In some variations, the first portion 152 of the conduit 150 may follow a generally straight path, and may be sized to extend along a substantial portion of the leg portion of the garment. However, the conduit may have any suitable shape and/or length. For example, the conduit may follow a curving path or a sinusoidal path (which may, for example, enable the conduit (and the one or more sensor assemblies accommodated therein) to stretch to accommodate stretching and flexing of the overall garment. As depicted in FIG. 2 and the cross-sectional view of FIG. 4, in some variations, the first portion of the conduit may function as a passage through which sensor assemblies 110 extend from the pocket 130 towards the foot portion 108 of the garment 102 before branching into channels 160 of the second portion 154 of the conduit. As shown in FIG. 2, the first portion 152 of the conduit 150 may be located distal to the pocket 130 and proximal to the foot portion 108 of the garment 102. The first portion of the conduit may comprise openings at a proximal end and a distal end to allow the sensor assemblies to pass through and into the other passages of the garment 102. The conduit 150 may be located on a lateral surface of the garment 102, such that when the user wears both and right and left sock, the conduits 150 do not face each other. Variations of the present invention that comprise a conduit 150 extending along a lateral side of the leg portion 106 may provide advantages to users. Generally, a side of a garment may experience less stretching, compression, and impact, etc. when worn on a user's foot—even when the user walks, runs, stretches, etc.—than a top or a sole of the garment. Therefore, sensor assemblies 110 situated in the left conduit, for example, may experience reduced impact and cyclical loading compared to fibers on the top and sole of the left garment when the garment is worn by a user. Thus, sensor assemblies 110 extending through the conduit 150 may be protected from impact and high degrees of repeated strain (in both tension and bending) when the garment 102 is worn on the user's foot. This protection may be particularly important, for example, when the garment 102 is worn over a long period of time. The conduit 150 may at least partially encase the sensor assemblies 110 as they extend down the leg portion 106 of the garment 102 and provide support to the electrical leads housed 112 of the sensor assemblies 110. This may help protect the leads 112 from failure such as due to excessive and repeated bending or stretching.

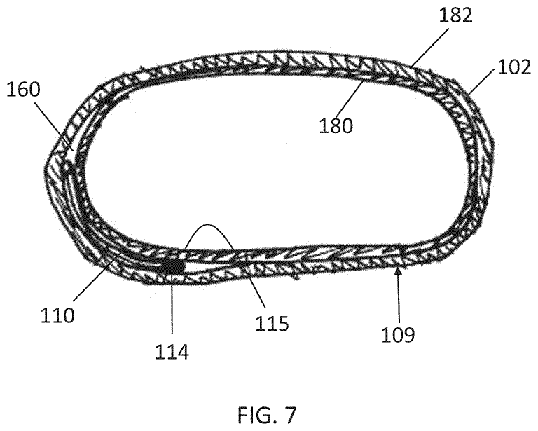

In some variations, while the first portion of the conduit may direct a bundled collection of sensor assemblies along a portion of the garment (e.g., leg portion), a second portion 154 of the conduit 150 may direct individual sensor assemblies 110 toward respective target sense locations. For example, from a central location in the leg portion of the garment (e.g., the first portion 152 of the conduit) to one or more target sense locations distributed throughout the foot portion of the garment. In order to distribute the sensor assemblies 110 to their respective target sense locations 107, the second portion 154 of the conduit 150 may comprise one or more channels 160 that branch off from the first portion 152 of the conduit. The channels 160 may direct the sensor assemblies 110 both laterally and distally within the garment 102. As shown in the variation depicted in FIG. 2, the second portion 154 of the conduit may comprise a plurality of channels 160 that branch from the first portion of the conduit 152 at or near the junction of the leg portion 106 and the foot portion 108 of the garment 102 to direct the sensor assemblies 110 to the target sensor locations 107 in the foot portion 108 of the garment 102. As depicted in FIG. 2, each channel 160 may be directed to, and terminate at, a respective target sense location 107 (e.g., 107a, 107b, 107c, etc.). For example, a first channel may direct a temperature sensor of a first sensor assembly to a first target sense location 107a, and a second channel may direct a temperature sensor a second sensory assembly to a second target sense location, etc. In some variations, as depicted in the cross-sectional view of FIG. 7, each channel may house one sensor assembly. FIG. 7 shows the lateral distribution of a single channel 160 comprising a single sensor assembly 110, within the foot portion of the garment. However, in another variation a single channel 160 may house any suitable number of sensor assemblies 110 (e.g., two, three, or more). As depicted in FIG. 2, one channel may be directed to each target sensor location 107. However, in another variation, multiple channels 160 may be directed to a single target sense location 107. In one variation, the garment may comprise six channels 160, each directed to one of six target sense locations 107. However, any suitable number of channels 160 may branch from the conduit 150 to a plurality of target sense locations 107. Further, the first or second portions of the conduit 150 may be reinforced by additional material such as plastic or flexible metal to add structure to the channels and prevent damage to the sensor assemblies 110.

Channels 160 may traverse and/or wrap around one side of the foot 108 toward the sole 109, or they may traverse and wrap around both sides of the foot. For example, all of the channels 160 may traverse the outer side of the user's foot to reach their respective target sense locations 107 on the sole of the foot. However, in another variation, a first subset of channels 160 may branch towards one side of the user's foot, while a second subset of channels 160 may branch toward the opposite side of the user's foot. It may be the case that the channels 160 are distributed equally along both the internal and external sides of the user's foot (for example, three channels may branch to the right side of the foot and three channels may branch to the left side of the foot). One benefit of distributing the channels 160 along two sides of the foot rather than situating all the channels 160 on one side of the user's foot is that the equal distribution may make the sock feel more balanced and thus more comfortable to the wearer (e.g., because the bulk of the sensor assemblies is distributed more evenly). However, any suitable distribution of channels may be used.

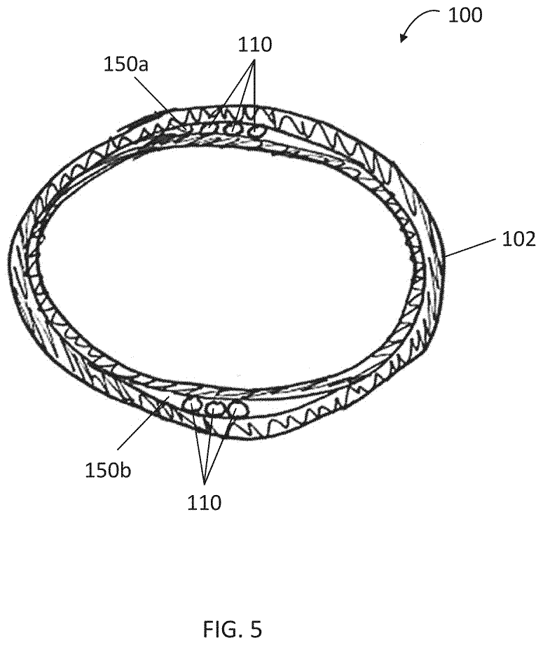

Further, the garment 102 need not contain channels 160 that branch off from the first portion 152 of the conduit 150. For example, the conduit 150 may direct the sensor assemblies 110 to the foot portion 108 and widen so that each sensor assembly 110 may reach a target sense location 107. In another variation, the garment 102 may comprise multiple (non-branching) conduits 150, each directing a sensor assembly 110 to a respective target sense location 107. As depicted in FIG. 5, the system may comprise two conduits 150. In other variations, the system may comprise any suitable number of conduits 150. For example, in FIG. 5, a first conduit 150a and second conduit 150b are situated on opposite sides of the garment 102. The first conduit 150a may be situated on a lateral region of the leg portion 106 of the garment 102, and the second conduit 150b, may be situated in a medial region of the leg portion 106 of the garment. Alternatively, the first conduit 150a may be located in a front portion of the leg portion and the second conduit 150b may be located in a back portion of the leg portion. In a variation of the system where the garment comprises two conduits, the conduits 150 may be evenly situated 180 degrees from each other in the garment 102. However, the conduits 150 may also be dispersed unevenly in the garment 102. In a variation of the system where the garment 102 comprises three conduits, the conduits 150 may be situated 120 degrees from each other. Any number of conduits may be situated in any suitable configuration within the garment 102. Each conduit may comprise one or more sensor assemblies 110, each directed to a target sense location 107. A system with two (or more) conduits 150 as depicted in FIG. 5 may provide the advantage of evenly distributing the bulk of the sensor assemblies 110. Further, multiple conduits 150 may reduce the need for sensor assemblies 110 to cross over the foot portion 108 of the device. This may allow the sensor assemblies 110 to be extended through the garment in a more direct fashion (i.e., requiring the sensor assemblies to traverse less of the garment to get to a target sense location.)

### Buffer Region

The garment may further comprise a compartmental buffer region 140 to contain excess length of sensor assembly 110, where the excess length may gather when the garment is relaxed (in its neutral state, or not stretched). The buffer region 140 may, for example, provide an advantage of protecting the sensor assemblies 110 from damage during use of the garment. Generally, when a user places her foot in the garment, she may pull the proximal portion of the garment 102 up her ankle and lower leg, thereby stretching and tensioning the garment 102. Other manipulations (e.g., folding, rolling, etc.) may similarly stretch and tension the garment. However, sensor assemblies 110 extending between the electronics system 130 and the target sense locations 107 may not be stretchable. For example, portions (e.g., the electrical leads 112 as described below) of the sensor assemblies 110 may break when tensioned. Therefore, to accommodate garment stretchability and reduce the likelihood of damage to the sensor assemblies when the garment is stretched, each sensor assembly 110 may be substantially longer than the distance or conduit length between the electronics system 130 and sensor assembly's respective target sense location 107 when the garment is in an unstretched or relaxed state (and at least slightly longer than the distance or conduit length between the electronics system 130 and the sensor assembly's respective target sense location 107 when the garment is tensioned (e.g., stretched to about 95% of its maximum stretched length). For example, in some variations, a sensor assembly 110 may be between about 0.5 cm and about 7 cm, between about 1 cm and about 6 cm, or between about 3 cm and about 5 cm longer than the length required to extend the assembly from the electronics system to a target sense location 107. Although one advantage of this excess length may be to help protect the sensor assembly from damage when the garment is stretched, in some variations, this excess length may additionally or alternatively advantageously provide sufficient length for manipulating the sensor assembly during manufacturing, such as when coupling sensor leads with the electronics system. For example, if a sensor assembly is already situated in the conduit, such extra length at a proximal end of the sensor assembly may be pulled out and manipulated when being soldered to the PCB as described in further detail below.

The buffer region 170 provides a place to contain this excess length when the garment and/or sensor assemblies 110 are not being stretched. In some variations, the excess length may be organized in a serpentine arrangement, which may, for example, extend and re-collapse into the serpentine arrangement as the garment is tensioned and released, respectively. As depicted in FIG. 2, the buffer region 140 may comprise a widened region at the proximal end of the conduit 150 distal to the pocket 170. In another variation, the pocket 170 may be oversized for the electronics system 130 to accommodate extra lengths of sensor assemblies 110, thereby functioning as a buffer region 140. However, the buffer region 140 can be defined by any suitable geometry. Further, although FIG. 2 depicts the buffer region 140 as being located near the pocket 170, the buffer region 140 may be located anywhere in the garment that is traversed by the sensor assemblies 110. For example, the buffer region 140 may be located at any point along the leg portion of the garment, or, the buffer region may be located near the junction of the first portion and the second portion of the conduit. In another example, the buffer region 140 may be located in the foot portion of the garment. It is also not necessary for the buffer region 140 to be a separate component from the conduit 150 or the pocket 170. The buffer region may be coextensive with either the pocket 170, the conduit 150, or both.

The garment may be constructed with various one or more passageways and/or openings designed to accommodate and support the components of the system used collect temperature and/or other sensor data. The one or more passageways and/or openings of the garment may be constructed in any suitable way. In one exemplary “1.5 layer” implementation, the garment includes a single primary layer (e.g. a “shell”), and the conduits(s), channels, buffer region, and pocket are individually knitted (in a “half layer”) into the outside of this primary knitted layer, such as in situ by a 3D flat knitting machine. In this implementation, the garment can thus define a “1.5-dimensional” garment with structures forming the conduit(s), channels, buffer region, and pocket.

In another exemplary “2 layer” implementation, the garment includes an inner layer and an outer layer, and the inner and outer layers are selectively knitted (or sewn) together to form the conduits(s), channels, buffer region, and pocket. In this implementation, the garment can thus define a 2-dimensional knitted garment with joined inner and outer structures forming the conduits(s), channels, buffer region, and/or pocket. For example, a garment may include a tube that is everted (e.g., one end folded into the opposing end) to form a double-layer garment that includes inner and outer layers. As another example, a first garment may provide an inner layer and a separate, second garment may provide an outer layer. The inner and outer layers may be stitched together around the mouth of the garment and around a toes region of the garment to preserve this arrangement of the inner and outer layers. The inner and outer layers may also be selectively stitched together to form the conduits(s), channels, buffer region, and/or pocket. The garment may thus define a “2 dimensional” garment.

In another exemplary “2.5 layer” implementation, the garment includes inner and outer full garment layers (two layers) that are knitted simultaneously and bridged with knitted fibers (a “half layer”) to form the conduits(s), channel(s), buffer region, and pocket. For example, a 3D flat knitting machine can simultaneously knit the inner and outer sock layers and knit bridging fibers between the inner and outer sock layers to form the conduits(s), channel(s), buffer region, and pocket. The garment can thus define a “2.5-dimensional” garment.

### Sensor Assembly

Sensor assemblies 110 may include sensors to obtain measurement data at one or more target sense locations and extend through the garment 102 to transmit sensor data from the sensors 114 to another location of the garment. For example, in a system comprising a garment 102 configured to be worn on the foot of a user, sensors 114 may be located in the sole 109 of the foot portion 108 of the garment in order to detect, for example, temperature at various locations along the foot of the user. As described in further detail below, electronic components for receiving, processing, storing, and/or transmitting data from the sensors 114 may be located in location(s) on the garment that are different from the measurement locations. For example, the sensors 114 may be located in the foot portion 108 of the garment 102, and the electronic components may be located in the leg portion 106. Thus, sensor assemblies 110 comprising sensors 114 may be used to electrically couple the sensors 114 to additional electronic components. As shown in exemplary FIGS. 6A-6C, each sensor assembly 110 may comprise a proximal end 116, a distal end 118 comprising a sensor 114, and one or more electrical leads 112 extending between the proximal end 116 and the sensor 114. The leads 112 may be exposed at the proximal end 116 of the assembly 110 to facilitate electrical coupling of the assembly 110 to the electronics system 130. The sensor 114 at the distal end of the sensor assembly 110 may be a temperature sensor comprising power and sense terminals. The signal transmitted by the sensor to the electronics system may be proportional to a local temperature at the target sense location 107 where the sensor 114 is situated. The signal may be any signal suitable to transmit sensor information, for example, a low-voltage analog signal. Further, any suitable variation of temperature sensor may be used. For example, the temperature sensor may be a thermistor, a thermocouple, a resistance based sensor, a semiconductor, or any other type of temperature sensor. Further, the sensors may not be temperature sensors. Sensors may be any suitable type of sensors for monitoring a patient, for example, a pressure sensor, a proximity sensor, a light sensor, an ultrasonic sensor, an infrared sensor, an acoustic sensor, or any other type of sensor.

Electrical leads 112 may be used to transmit data from the sensor 114 to the proximal end 116 of the sensor assembly 110. The electrical leads 112 of the sensor assembly 110 may extend from the sensor 114 to the proximal end 116 of the assembly 110. The leads 112 may be encased by insulated coating. As depicted in FIG. 6B, one or more longitudinal or axially-oriented packing fibers 126 may be arranged along the length of the electrical leads 112 within the sensor assemblies 110. At least a portion of the sensor assembly may be covered by a sheath. For example, the sheath may include one or more wrapping fibers 111 wrapped circumferentially about the sensor assembly 112 to encase and support the elements of the assembly 110. In one variation, the fibers may be wrapped helically about the sensor assembly 110. However, the sheath need not be formed by wrapping fibers circumferentially about the sensor assembly 110, and may be of any suitable construction. For example the fibers may be braided to encase the sensor assembly 110, or may extend parallel to the sensor assembly. Further, the sheath may be made of any suitable material, and may encase the sensor assembly 110 in any suitable way. For example, the sheath may be made of a plastic or polymer material, braided or extruded tubing, intermittent bands, or any other suitable material. The wrapping fibers 111 may terminate between the sensor 114 and the end of an engagement feature 115 at the distal end of the sensor such that the engagement feature 115 is physically accessible just beyond the sensor 114.

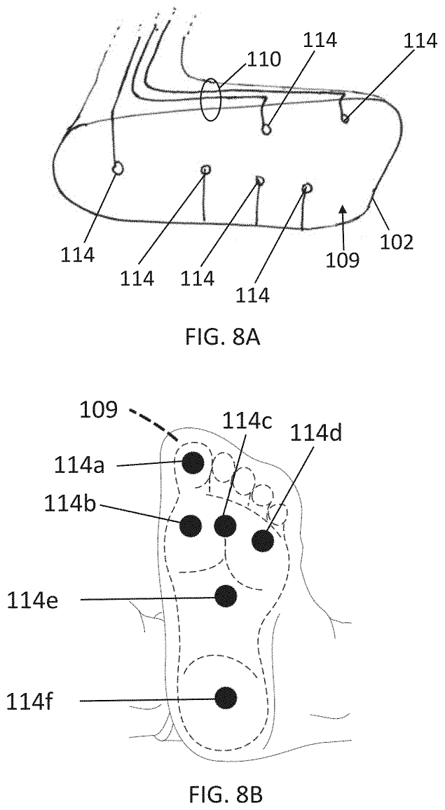

Sensors 114 of the sensor assemblies 110 may be arranged to collect data to meaningfully monitor a user. For example, in a system for monitoring the foot health of a user, sensors 114 may be arranged in the sole of a garment configured to be worn on the foot of the user. Sensors 114 may be located at target sense locations 107 in the sole 109 of the foot portion 108 of a garment in a pattern that optimizes data collection of temperature values from pressure points in a user's foot. For example, FIGS. 8A and 8B depicts an exemplary pattern of the sensors 114 located in the sole 109 of the foot portion 108 of the garment 102. Each sensor 114 may be aligned with a target sense location 107 in the sole 109 of the garment 102. In some variations, at least some of the target sense locations 107 may correspond to locations of a user's foot that typically experience high pressure when the user it active (e.g. walking or standing). For example, the sensor arrangement may be constructed so as to arrange the one or more temperature sensors 114 on the sole region of the garment. FIG. 8A depicts an exemplary arrangement of six sensors 114 located at six target sense locations 107 on the sole 109 of the garment 102. In the exemplary variation as shown in FIG. 8B, the temperature sensor pattern on the sole 109 of the garment 102 may include six temperature sensors 114 (114a-114f). One temperature sensor 114a may be positioned in a first region of the garment 102 configured to contact an Ossa digit of the foot, or toe. For example, the temperature sensor 114a may be configured to contact skin overlying the first Ossa digit (“big toe”) of the foot. Three temperature sensors 114b, 114c, and 114d may be positioned in a second region of the garment 102 configured to contact skin overlying the boundary of the phalanges and the metatarsals of the foot. For example, the temperature sensors 114b, 114c, and 114d may be configured to contact skin proximate the first (most medial) metatarsal, the third metatarsal, and the fifth metatarsal, respectively. Additionally, another temperature sensor 114e may be positioned in a third region of the garment 102 configured to contact skin overlying the boundary of the metatarsals and the tarsals of the foot. Furthermore, another temperature sensor 114f may be positioned in a fourth region of the garment 102 configured to contact skin overlying the heel of the foot. The configuration of temperature sensors may be biased for a left foot in instances in which the garment is configured to be placed on a left foot, while the configuration of temperature sensors may be biased for a right foot in instances in which the garment is configured to be placed on a right foot. Although FIG. 8B depicts six target sense locations, any suitable number of target sense locations 107 may be used (e.g., one, two, three, four, five, seven, or more than seven, etc.). Further, the system described here may comprise any suitable number of sensors 114. Although FIG. 8B depicts an arrangement where one sensor 114 aligns with one target sense location 107, as stated above, multiple sensors 114 may be aligned with a single target sense location 107.

### Engagement Feature

Furthermore, in some variations the sensor assembly may include an engagement feature for facilitating installation of the sensor assembly in the garment. As further described below, the engagement feature may, for example, be temporarily coupled to an insertion tool that is used to guide the installation of the sensor assembly in a conduit or channel. In some variations, the engagement feature may include a loop. For example, as depicted in FIG. 6A, a fiber 117 with a loop 115 at a distal end may be arranged to extend along the length of the electrical assembly 110. The loop 115 may be formed in any of various suitable manners. For example, a fiber 117 extending along the length of the sensor assembly may be formed into a loop 115 at a distal end, wherein at least a portion of the loop 115 extends past the distal end of the sensor 114. In one example, the loop 115 may be formed by extending one end of the fiber 117 along the length of the sensor assembly 110, and then looping the fiber 117 back along the sensor assembly in the opposite direction, leaving a middle portion of the fiber extended past the sensor 114. The loop 115 may extend past the sensor 114, and may be coupled to the sensor 114, for example, with adhesive 119 (e.g., epoxy). However, the loop may be secured to the sensor in any suitable way. In another example, the loop may be coupled to the wrapping fibers or packing fibers rather than to the sensor.

In some variations, the engagement feature may include a ring (e.g., a flexible ring such as silicone or other elastomer) secured at the distal end of the sensor assembly, such as coupled to the sensor, packing fiber(s), etc. In another variation, the engagement feature may include a hook secured at the distal end of the sensor assembly. The engagement feature may include a snap or hook and loop-type fastener material, or the like secured at the distal end of the sensor assembly. The engagement feature may additionally or alternatively include a magnet at the distal end of the sensor assembly. In an embodiment in which the engagement feature is a magnet, the sensor assembly 110 may be guided through the garment using an external magnet. Furthermore, it should be understood that the engagement feature may be located at any location along the sensor assembly and need not be located at the distal end. Further, each sensor assembly may comprise multiple engagement features at a distal end or along the length of the sensor assembly.

The engagement feature of each sensor assembly may be secured to a channel 160 in order to keep the sensor 114 in place at the target sense location 107. For example, the engagement feature 115 may be threaded through a fiber of the garment 102 within the channel 160. In other variations, the channel 160 of the garment may contain a fiber or protrusion for the engagement feature 115 to be attached to (e.g., tied). In another variation, the engagement feature 115 may be sewn into the fibers of the garment 102. However, the sensor need not comprise an engagement feature. The sensor may be held in place at the target sense location 107 by restrictive forces of the channel 160 on the sensor assembly 110. In another variation, the sensor assembly 110 may be directly sewn into the channel 160, or attached to the channel using adhesive.

Furthermore, in some variations, a sensor assembly 110 may include additional elements arranged in various ways, such as described in U.S. Patent Publication No. 2018/0087192, which is incorporated herein in its entirety by this reference.

### Housing and Electronics System

As described above, a system for monitoring a user may comprise an electronics system 130 to receive, and communicate data. The electronics system may receive data, for example, from sensors 114 (e.g., temperature sensors) and transmit the data to an external processing system. At least some of the components of the electronics system 130 may be encased by a housing 139 configured to secure and protect the components. The housing 139 may include at least one cavity for receiving a proximal end 116 of the sensor assembly 110 as further described below, and/or housing other various components. The housing 139 containing the electronics system 130 may be situated in a garment 102 configured to be worn on the foot of a user, as shown in FIG. 2 and FIG. 3A. As shown in FIG. 3A, the electronics system 130 may be retained by the pocket 170 of garment as described above. Additionally or alternatively, the housing 139 may be coupled to the garment 102 with an adhesive or mechanical fasteners (e.g., rivets, snaps, etc.), by being inserted into a pocket or other receptacle on the garment, sewn to the garment, or in any suitable manner. The proximal end 116 of each sensor assembly 110 may be coupled to the electronics system 130 via electrical leads 112. This electrical connection may allow the sensors 114 at the distal end 118 of the assembly 110 to transmit data to the electronics system 130, and the electronics system 130 to supply power to the sensors 114. Each sensor assembly 110 may comprise any suitable number of leads. For example, as depicted in the exemplary variation of FIG. 6A, each sensor assembly may comprise two electrical leads 112. The electrical leads 112 of the sensor assemblies 110 may be coupled to the housing and/or one or more of the components of the electronics system contained by the housing. In another variation, the housing may be reversibly or removably connected to the sensor leads (e.g., the sensor leads may be disconnected via a connector interface) such that the housing and electronics system are removable from the pocket after being disconnected from the sensor assemblies.

The housing 139 may be substantially sealed (e.g., hermetically sealed or waterproofed) to protect the contents of the housing from environmental conditions (e.g., when the garment is washed or worn, when the user is sweating, etc.). For example, a sealant adhering to the housing 139, such as ultraviolet glue, silicone, other epoxy or polymer, etc. may be used to seal any openings in the housing 139, and may help fix the proximal end 116 of the sensor assemblies 110 and/or other components within the housing 139. As another example, the housing 139 may include one or more components that may be coupled together (e.g., with a suitable mechanical interfit, fasteners, weld, etc.). The joint between coupled housing components may be sealed by epoxy or other suitable sealant. In some variations, the one or more components of the housing may be formed at least in part through injection molding.

Generally, the components of the electronics system may receive data from the sensor assemblies 110, supply power to the sensors 114 of the sensor assemblies 110, store information collected from the sensor assemblies, 110, and/or transmit data to an external processing system. The electronics system 130 in the exemplary variation depicted in FIG. 3A may comprise a power source 132, a controller 134, a wireless communication module 136, and a PCB 138. As depicted in FIG. 1B, the components of the electronics system may interact with the one or more sensor assemblies 110 to process the data transmitted from the sensors (114a, 114b, and 115c) to the electronics system. In some variations, the electronics system may further include one or more additional activity or other sensors (e.g., an accelerometer, a gyroscope, or an inertial measurement unit). One or more of these components may be arranged on one or more electronic circuit boards (e.g., PCBA), which in turn be mounted to the housing

The electronics system may include one or more power sources 132, which may function to provide electrical power to the power source 132, a controller 134, a wireless communication module 136, PCB 138, and/or any other electrical components. For example, the power source 132 may include one or more batteries. In some variations, the power source 158 may be rechargeable such as through wireless charging methods (e.g., inductive charging, RF coupling, etc.) or by harnessing kinetic energy such as that generated through motion (e.g., when the user walks while wearing the garment).

The controller 134 of the electronics system may receive and collect temperature data from the sensor 114 via the electrical leads 112 of the sensor assembly 110, and may locally store this data, before transferring the data to the wireless communication module 136. One or more processors 131 and memory devices 133 may cooperate to provide the controller 134 for the electronics system 130. For example, the processor 131 (e.g. a CPU) may receive data from one or more sensors 114, and the sensor data may be stored in one or more memory devices 133. The data received from the sensors of the sensor assemblies may be temperature data, for example. That data may be transmitted in any suitable form, for example via voltage readings from the sensor. Sensor data may be stored in one or more memory devices 133. In some variations, the processor 131 and memory 133 may be implemented on a single chip, while in other variations they can be implemented on separate chips.

The controller 134 can operate in an inactive state and in an active state. The controller may, for example, toggle between the inactive state and the active state based on user input (e.g., pressing of a button) and/or sensor data (e.g., from activity sensors, processing of temperature data, etc.) suggesting placement of the garment on a user. In the inactive state, the controller may be in a “sleep” mode (e.g., to conserve energy in the power source 132). In the active state, the controller may be in an “awake” mode in which sensor data is received, processed, and/or stored in the memory device 133 for use in monitoring for inflammation. For example, in its active state, the controller may scan at least some of the temperature sensors to receive and store temperature measurement data (e.g., periodically, such as every second, every 10 seconds, every 30 seconds, every minute, every hour, or other suitable interval). Generally, the controller may operate in the inactive state when there is an indication that the garment is not being worn by the user, and may operate in the active state when there is an indication that the garment is being worn by the user. In some variations, the controller may be similar to the controller described in U.S. Patent Publication No. 20170188841, incorporated by reference above.

The controller 134 may regularly offload data via the wireless communication module 136 to an external computation system for processing. The wireless communication module 136 may, for example, export the data transmitted by the sensors 114 and collected by the controller 134 to any suitable external computation system. The external computation system 190 may be, for example, a mobile computing device (e.g., mobile telephone, tablet, smart watch), laptop, desktop, or other suitable computing device. The external computation system 190 may be executing a native application for presenting sensor data (and/or the results of analysis thereof) through a user interface to a user. Additionally or alternatively, the wireless communication module 136 may be configured to communicate to one or more networked devices, such as a hub paired with the system, a server, a cloud network, etc.

The wireless communication module 136 may communicate via a wireless network (e.g., through NFC, Bluetooth, WiFi, RFID, or any type of digital network that is not connected by cables). For example, devices may directly communicate with each other in pairwise connection (1:1 relationship), or in a hub-spoke or broadcasting connection (“one to many” or 1:m relationship). As another example, the devices may communicate with each other through mesh networking connections (e.g., “many to many”, or m:m relationships), such as through Bluetooth mesh networking. Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), or any other suitable communication protocol. Some wireless network deployments may combine networks from multiple cellular networks or use a mix of cellular, Wi-Fi, and satellite communication.

The external computational system may interpret the data received from the wireless communication module, and may notify the user based on the data. For example, the external computational system 190 may interpret temperature data from sensors 114 located at target sense locations on the sole of the garment and notify a user of developing foot problems. As described in U.S. Patent Publication No. US 2017/0188841, incorporated by reference above, data comparison between a right and left garment may be used to interpret temperatures as indicative of inflammation or other medical complications in the user's feet. For example, when a garment is worn by a user on his left foot, a controller can regularly collect temperature values across the sole of the user's left foot through a left set of temperature sensors integrated into the left garment. Similarly, a controller in the right garment worn on the user's right foot can regularly collect temperature values across the sole of the user's right foot through a right set of temperature sensors integrated into the right sock. These controllers can regularly offload temperature data read from these temperature sensors to the user's smartphone (or other local computing device) executing a native application that calculates temperature differences between like temperature data originating from temperature sensors in similar locations between the left and right socks and that notifies the user (or an affiliated care provider) of possible inflammation in one or both of the user's feet responsive to such differences. Alternatively, such data may be communicated to one or more remote computing devices (e.g., server) for similar analysis.

The PCB 138 may support the components of the electronics system 130, such as the power source 132, the controller 134, and the wireless communication module 136. The PCB 138 may include traces that electrically couple the sensor assemblies 110 to the components of the electronics system 130, such as the power source 132 and controller 134. Electrical leads 112 of the sensor assemblies 110 may be connected to the electronics system 130 by coupling the leads to the PCB 138. However, the electrical leads may be coupled to the components of the electronics system in any suitable way. For example, the electrical leads may be soldered to the PCB, may be bound by adhesive, or may be retained by ports in the housing 139.

### Methods for Manufacturing

Described below are methods relating to manufacturing a garment configured to support components of a system that collect sensor data from a user. In some variations, the garment may be configured to be worn on the foot of a user, and may comprise one or more sensors located in a foot portion of the garment to measure local temperatures (and/or other biometrics) of a user's foot. Generally, a method of making a garment for monitoring a user may comprise passing a sensor assembly through a garment, electrically coupling the sensor assembly to an electronics system, and securing the electronics system to the garment. In some variations, the garment may be configured to be worn on the foot of a user, and may comprise a leg portion and a foot portion. For example, the garment may be any of the garment variations described, or the like. Specifically, the garment may comprise a conduit configured to direct one or more sensor assemblies to target sense locations in a sole of the foot portion of the garment. A first portion of the conduit may be situated in the leg portion of the garment, and a second portion of the conduit may comprise channels that branch from the first portion of the conduit into the foot portion of the garment. The leg portion of the garment may comprise a pocket and a first portion of the conduit. The foot portion of the garment may comprise the second portion of the conduit comprising one or more channels directed to one or more target sense locations. Passing a sensor assembly through a garment may comprise extending the sensor assembly sensor-end first into the pocket and through the first portion of the conduit. Passing the sensor assembly through the garment may further comprise extending the sensor assembly from the first portion of the conduit and into a channel of the second portion of the conduit directed to a target sense location. The method may further comprise securing the sensor assembly to the target sense location. The method may further comprise electrically coupling the sensor assembly to an electronics system. The sensor assembly may comprise a sensor at a distal end, and a set of electrical leads extending from the sensor to the proximal end of the assembly. The leads may be attached to components of the electronics assembly. The method may further comprise placing the electronics assembly into a pocket of the garment.

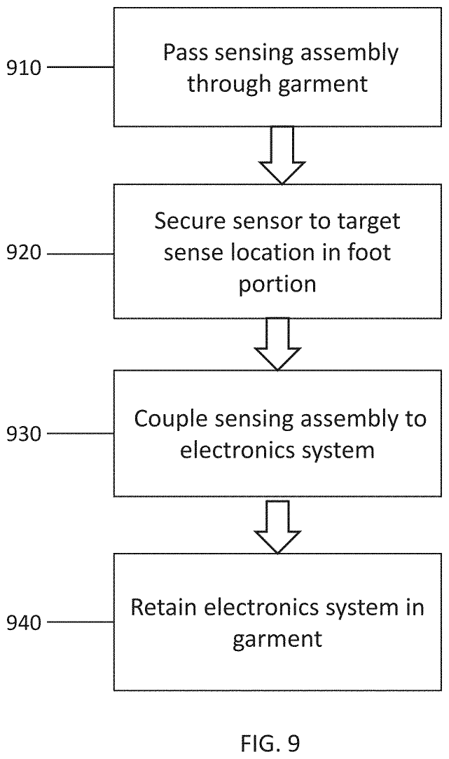

FIG. 9 is a schematic representation of methods for manufacturing a garment to monitor a user. The method may comprise passing a sensor assembly through a garment 910 configured to be worn on the foot of a user; securing a sensor of the sensor assembly to a target sense location 920 on a foot portion of the garment; coupling the sensor assembly 930 to an electronics system; and retaining the electronics system in the garment 940. For example, in some variations, a method for making a garment may include passing at least one sensor assembly at least partially into a conduit of a garment, where the sensor assembly may include at least one sensor (e.g., temperature sensor), positioning the sensor in the conduit at a target sense location, coupling a distal portion of the at least one sensor assembly to the garment such that the sensor is secured at the target sense location, and coupling a proximal portion of the at least one sensor assembly to an electronics system.

Although the method is described in terms of threading or passing one sensor assembly into a garment, this method may be repeated to thread or pass any suitable number of sensor assemblies into a garment. A sensor assembly may comprise a proximal end, a distal end comprising a sensor, and one or more electrical leads extending from the proximal end to the sensor. The sensor may be a temperature sensor, pressure sensor, or the like.

Passing a sensor assembly through a garment 910 functions to situate the assembly within the garment to direct the sensor end of the assembly to a target sense location. The method may comprise passing the assembly through the garment sensor-end first. In some variations, the garment may comprise a leg portion comprising a pocket, a buffer region, and a conduit, and a foot portion comprising one or more channels, each directed to a target sense location on the foot portion. The pocket may comprise one or more openings through which the sensor assembly may enter and exit. The conduit may extend along the length of the leg portion, directing the sensor assembly distally through the garment. The conduit may be configured to direct the sensor assemblies from the pocket to the channels in the foot portion of the garment. The conduit may comprise a top opening and a bottom opening to allow the sensor assemblies to enter and exit the conduit. The channels may diverge from the conduit in the leg portion and into the foot portion of the garment. The channels may be directed to a target sense location in the sole of the foot portion of the garment. Each channel may be directed to a target sense location on the sole of the foot portion of the device. In these variations, the method may comprise passing a sensor assembly through the pocket and buffer region of the leg portion of the garment. The method may further comprise passing a sensor assembly through the conduit in the leg portion of the garment. The method may further comprise passing the sensor assembly from the conduit in the leg portion of the garment into one of a plurality of channels in the foot portion of the garment. The method may further comprise positioning the sensor in the channel at a target sense location in the sole of the foot portion of the garment. In some variations, the sensor assembly may be manipulated through the pocket, buffer region, conduit, etc. by coupling an engagement feature (e.g., as described elsewhere herein) of the sensor assembly to an installation instrument such as a long needle (e.g., hooked onto a crochet needle). The tip of the instrument may be fed through the passageways of the garment and advanced to a target sense location (e.g., at the end of a channel).

The method may further comprise securing the sensor assembly to a target sense location, which functions to help the sensors collect temperature data at one or more desired locations. For example, it may be desirable to collect data from pressure points on a user's foot, and securing the sensors to these locations in a garment may aid in the collection of useful data. Further, sensor assemblies that do not stay in place may be uncomfortable for the user. For example, the sensor may move or bunch within the channel. Therefore, it may be beneficial to secure the sensor end of each sensor assembly to a target sense location. Each sensor assembly may comprise an engagement feature extending past the sensor of the sensor assembly. The engagement feature may, for example, be comprised of an axial fiber formed into a loop at the distal end of the sensor, wherein the engagement feature extends past the distal end of the sensor. Other variations of engagement features are described above.

The method may comprise securing the sensor in place at a target sense location within the channel. Securing the sensor at a target sense location may comprise attaching the engagement feature at the distal end of the sensor to the garment. The engagement feature, for example, a loop may be attached to the channel using any suitable method. For example, the method may comprise threading the loop portion through a fiber of the garment defining the channel. In another variation, the method may comprise hooking the loop onto a protrusion in the channel. In other variation, the method may comprise tying the loop to a fiber situated within the channel. In another variation, the method may comprise stitching the sensor end of the sensor assembly to the garment.

In some variations, sensor assemblies might not be configured to stretch in the way that knitted fibers of a garment might. For example, the electrical leads, packing fibers, or other components of the sensor assemblies might not be flexible in one or more directions. Therefore, in order to allow the user to manipulate the garment (for example, when the user pulls the garment onto his or her foot), it may be beneficial for sensor assemblies to have some length in excess of that strictly needed for the assemblies to extend from the electronics system to the target sensor location. The method may further comprise trimming the sensor assemblies to a length such that the proximal ends of the assemblies may extend past the pocket when the garment is maximally stretched longitudinally. The excess length of the sensor assemblies may be passed into a buffer region of the garment. Once the sensor assemblies are connected to the electronics system, excess lengths of sensor assembly can be drawn into the pocket and into the buffer region in the leg portion of the garment, and the electronics system can be inserted into the pocket.

The method may further comprise coupling the sensor assembly to an electronics system. In order for data to be transmitted from the sensors in the foot portion of a garment to the electronics system located elsewhere in the garment, sensor assemblies may need to be coupled to an electronics system. Coupling the sensor assembly to the electronics system may comprise attaching the proximal end of the sensor assembly to one or more components of the electronics system. As described above, the sensor assembly may comprise electrical leads extending from the proximal end of the assembly to the sensor at the distal end of the assembly. The electrical leads may be exposed at the proximal end of the sensor. Alternatively, the method may comprise exposing the leads at the proximal end of the sensor. For example, the method may comprise pushing back fibers wrapped around the leads of the sensor assembly to expose the leads. In other variation, the method may comprise cutting or tearing an outer wrapping of the sensor assembly to expose the leads. Coupling the sensor assembly to the electronics system may comprise coupling one or more leads at the proximal end of the sensor assembly to one or more components of the electronics system. The components of the electronics system may include a power source, a controller, a wireless communication module, and a PCB. The method may comprise attaching a first lead of the sensor assembly to the power source. The first lead may be connected to a power terminal of the sensor at the distal end of the sensor assembly. Connecting the first lead to the power source of the electronics system may operate to supply power to the sensor. The method may further comprise attaching a second lead of the sensor assembly to the controller. The second lead be attached to a sensor terminal of the sensor at the distal end of the sensor assembly. Connecting the second lead to the controller may allow the sensor to transmit data (e.g. temperature data) from the sensor to the controller.

In some variations, the method may further comprise encasing the components of the electronics system in a housing. This step may be performed before or after the leads of the sensor assembly are connected to the components of the electronics system. Where the components of the electronics system are encased in a housing before the leads at the proximal end of the sensor assemblies are coupled to the electronics system, coupling the leads to the electronics system may comprise inserting the leads into ports in the housing. The method may further comprise sealing the components of the electronics system and sealing the connection of the leads to the electronics system. Sealing the components electronics system may comprise covering the housing of the electronics system with adhesive or sealant. Any suitable method may be used to seal the components of the electronics systems. For example, the housing may be wrapped in any suitable sealing material or may be wrapped in fibers. Sealing the electronics system may further comprise sealing openings in the housing that accommodate the leads with adhesive to protect the leads and secure the connection between the leads and the components of the electronics system. Sealing the electronics system may have the benefit of providing an electronics system that has been waterproofed (e.g., allowing the garment to be washed as needed, protecting the electronics system against perspiration, etc.).

The method for manufacturing a garment may further comprise retaining the electronics system in the garment. This may comprise placing the electronics system (and/or housing in which the electronics system is disposed) inside a pocket situated in the leg portion of the garment. As described above, the pocket may be located in the leg portion of the garment proximal to the conduit. Placing the electronics system in the pocket may comprise inserting the electronics systems through an opening in the pocket. The opening in the pocket may situated in an outer layer of the garment. Alternatively, the opening may be situated in an inside layer of the garment. In another variation, the opening may be between the layers of the garment. For example, the opening may be a gap in the stitching of the layers of the garment that allows the electronics component to be inserted between the layers near the mouth of the garment. Retaining the electronics system with the garment may comprising securing the electronics system within the pocket. This may comprise closing or shrinking the opening of the pocket. A method of shrinking the opening may comprise shrinking the garment. The garment may be knitted with unwashed fibers to an oversized dimension, which may ease assembly of the sock, such as threading smart yarn segments through the device and inserting the electronics system into the pocket. Once the sensor assemblies and the electronics system are assembled into the garment, the method may comprise washing the garment, which may cause the garment fibers to constrict, thereby shrinking the garment to a target dimension and improving retention of the electronics system in the pocket. Shrinking may also have the benefit of constricting the conduit and the channels to tighten around the sensor assemblies. This may allow the garment to more effectively support the sensor assemblies. Alternatively, the method of securing the electronics system within the pocket may not comprise shrinking the garment.

However, the electronics system may be retained in the pocket by any suitable method. For example, the pocket may comprise an elastic component that may stretch to allow the electronics system to be inserted into the pocket, but make it difficult for the electronics system to fall out of the pocket. The method of securing the electronics system may comprise placing the system in the pocket, and allowing the elastic to retain its shape. In another variation, the method may comprise operating a closure or seal of the pocket to secure the electronics system. For example, the pocket may comprise an opening with a zipper, snap, magnet, or any suitable mechanism to aid in securing the electronics system in the pocket. In another variation, the method of securing the electronics system in the pocket may comprise sewing the pocket shut after the electronics system is inserted into the pocket.

Methods for manufacturing a garment to be worn on the foot of a user may further include knitting a garment that defines: a pocket arranged on a side of the garment proximal a mouth of the garment and configured to receive an electronics system; a conduit extending along a side of the garment from the pocket toward a foot portion of the garment, approximately parallel to a longitudinal axis of the garment; and one or more channels, each channel extending from the conduit into the foot portion of the garment and terminating at a corresponding target sense location on a sole of the foot portion of the garment. Manufacturing a garment, for example, by knitting, sewing, weaving, or any other suitable method, may be automated (e.g., executed by a knitting machine).

The foregoing description, for purposes of explanation, used specific nomenclature to provide a thorough understanding of the invention. However, it will be apparent to one skilled in the art that specific details are not required in order to practice the invention. Thus, the foregoing descriptions of specific variations of the invention are presented for purposes of illustration and description. They are not intended to be exhaustive or to limit the invention to the precise forms disclosed; obviously, many modifications and variations are possible in view of the above teachings. The variations were chosen and described in order to explain the principles of the invention and its practical applications, they thereby enable others skilled in the art to utilize the invention and various variations with various modifications as are suited to the particular use contemplated. It is intended that the following claims and their equivalents define the scope of the invention.

1. A system for monitoring a user, the system comprising:
a garment configured to be placed on a foot of the user, wherein the garment comprises:
at least one conduit extending to a target sense location in a foot portion of the garment;
at least one sensor assembly arranged at least partially in the conduit, wherein the at least one sensor assembly comprises a temperature sensor located at the target sense location; and
a wireless communication module configured to communicate temperature data from the temperature sensor to a computing device.

2. The system of claim 1, wherein the garment further comprises a leg portion, and wherein a first portion of the conduit is in the leg portion of the garment, and a second portion of the conduit is in the foot portion.

3. The system of claim 2, wherein the second portion of the conduit comprises a plurality of channels diverging from the first portion of the conduit.

4. The system of claim 3, wherein the garment comprises a plurality of sensor assemblies arranged at least partially in the conduit, and wherein the plurality of channels comprises a first channel housing a first sensor assembly and a second channel housing a second sensor assembly.

5. The system of claim 4, wherein the first channel directs a temperature sensor of the first sensor assembly to a first target sense location, and wherein the second channel directs a temperature sensor of the second sensor assembly to a second target sense location.

6. The system of claim 3, wherein a first subset of the channels extends along a left side of the foot portion, and a second subset of the channels extends along a right side of the foot portion.

7. The system of claim 1, wherein the garment comprises a plurality of target sense locations arranged in a pattern on a sole region of the foot portion of the garment.

8. The system of claim 7 wherein at least one target sense location is in an ossa digit region of the garment, at least one target sense location is between a phalange region and a metatarsal region of the garment, at least one target sense location is between the metatarsal region and a tarsal region of the garment, and at least one target sense location is in a heel region of the garment.

9. The system of claim 1, wherein the garment further comprises a compartment housing excess length of the at least one sensor assembly.

10. The system of claim 1, wherein the at least one sensor assembly comprises a proximal end, a distal end comprising the sensor, and at least one electrical lead extending between the proximal end and the sensor.

11. The system of claim 10, wherein the at least one sensor assembly further comprises at least one axial fiber aligned with the at least one electrical lead.

12. The system of claim 11, wherein the at least one sensor assembly further comprises at least one wrapping fiber surrounding the at least one electrical lead and the at least one axial fiber.

13. The system of claim 10, wherein the proximal end of the sensor assembly is coupled to an electronics system via the at least one electrical lead.

14. The system of claim 10, wherein the at least one sensor assembly further comprises an engagement feature.

15. The system of claim 14, wherein the engagement feature comprises a loop portion coupled to the distal end of the at least one sensor assembly.

16. The system of claim 15 wherein the loop portion is coupled to the garment proximate the target sense location.

17. The system of claim 15, wherein the loop portion comprises an axial fiber formed into a loop that extends distal to the sensor.

18. The system of claim 1, wherein the garment further comprises a pocket.

19. The system of claim 18, wherein the pocket contains an electronics system encased in a housing, wherein the electronics system comprises at least one of a power source, a controller, and the wireless communication module.

20. A method of making a garment for monitoring a user, the method comprising:
passing at least one sensor assembly at least partially into a conduit of a garment, the conduit extending to a foot portion of the garment, wherein the sensor assembly comprises a temperature sensor;
positioning the temperature sensor in the conduit at a target sense location in the foot portion of the garment;
coupling a distal portion of the at least one sensor assembly to the garment such that the temperature sensor is secured at the target sense location; and
coupling a proximal portion of the at least one sensor assembly to an electronics system.

21. The method of claim 20, wherein passing the at least one sensor assembly into the conduit comprises passing the at least one sensor assembly through a first portion of the conduit in a leg portion of the garment, and into a second portion of the conduit in the foot portion of the garment.

22. The method of claim 21, wherein the second portion of the conduit comprises a plurality of channels diverging from the first portion of the conduit.

23. The method of claim 22, wherein passing the at least one sensor assembly into the second portion of the conduit comprises passing the at least one sensor assembly into one of the plurality of channels.

24. The method of claim 20, wherein the garment comprises a plurality of target sense locations arranged in a pattern on a sole region of the foot portion of the garment.

25. The method of claim 24, wherein at least one target sense location is in an ossa digit region of the garment, at least one target sense location is between a phalange region and a metatarsal region of the garment, at least one target sense location is between the metatarsal region and a tarsal region of the garment, and at least one target sense location is in a heel region of the garment.

26. The method of claim 20, wherein the at least one sensor assembly comprises an engagement feature, and wherein coupling the distal portion of the at least one sensor assembly comprises coupling the engagement feature to the garment proximate the target sense location.

27. The method of claim 20, wherein the engagement feature comprises a loop portion.

28. The method of claim 20, further comprising substantially sealing the electronics system in a housing.

29. The method of claim 28, further comprising placing the housing into a pocket in the garment.

30. The method of claim 29, further comprising shrinking the garment around the housing.
